(19) United States
(12) Patent Application Publication
(10) Pub. No.: US 2008/0082073 A1
(43) Pub. Date: Apr. 3, 2008

(54) Absorbent article with integrated ears

(76) Inventors: Stacy Driskell, Royersford, PA (US); Arginnys Soto, Yardley, PA (US)

Correspondence Address: GOSZ AND PARTNERS LLP, ONE STATE STREET, BOSTON, MA, 02109, US

(21) Appl. No.: 11/528,787

(22) Filed: Sep. 28, 2006

(51) Int. Cl. A61F 13/15

(52) U.S. Cl. 604/385.22; 604/385.3

(57) ABSTRACT

The invention discloses an integrated absorbent chassis for an absorbent article. The back sheet of the disclosed absorbent article is made from a single sheet of composite material that has elastomeric characteristics in predetermined regions. For example, the ear portions of the article are extendable for a better fit of the article. Other zones such as leg areas may also be elasticized if required.

### BACKGROUND OF THE INVENTION

The present invention relates to the field of absorbent articles for containing body exudates. More particularly, the invention relates to an hourglass-shaped absorbent article with a single piece chassis.

Disposable absorbent articles have long been used for the absorption of various kinds of body exudates. They have been used in various forms ranging from diapers for babies, to adult incontinence articles, to feminine health care products. Today, most of these articles employ an absorbent pad sandwiched between a liquid permeable sheet and a liquid impermeable sheet. These articles may take various shapes and sizes depending upon their need and applicability. For example, an article may have a pad-type structure when used for feminine health care products. The article may be hourglass-shaped for use as a baby diaper, or it may take the shape of a pant when it is used for toilet training young children, or as an incontinence device for adults having difficulty controlling their bladder.

Considerable effort has been expended in recent years to increase the ease of use of the absorbent articles. This effort has been directed towards increasing the functionality of the article, for example by improving absorption, allowing the retention of multiple insults, enabling reduced leakage from the article, enhancing ease of use and wearer comfort, by improving fit and minimizing skin irritation. Many efforts have also been made for designing a better-looking product, by using printed back sheets and reducing the product's bulkiness. Other innovations include features such as visual indicators of ‘time to change’ the article, ‘glow in the dark’ articles and articles with skin conditioners, etc.

Since an absorbent article is usually worn throughout the day, it is very important not only to minimize the skin irritation that may be caused by the article, but also to maximize the fit of the article on the body so as to increase the comfort of the wearer. Ease in wearing, cozy fit for long periods of time, and freedom in body movement are just some of the features desired from such a product.

Diapers are one type of absorbent article that have evolved over time from bulky, uncomfortable, leak prone, rectangular articles, to lightweight, long lasting, and appealing products. One important area where significant development has occurred in the field of diapers is in the way the diaper is fitted to the body. The hourglass design is one such innovation that was adopted by the industry in the 1980s. In the hourglass configuration, the diaper has a near rectangular crotch region. Above and below this region (i.e. at the shorter side of the rectangle) are relatively broad front and rear portions of the diaper. These sections together cover the front (i.e. the area above the crotch and below the stomach) and back portion (i.e. the bottom) of the wearer. The top edge of these sections (i.e. front and rear) together form the waist portion of the diaper. In order to have better coverage of the side portion of the body when a diaper is worn, these sections are progressively being redesigned with flaps projecting outwardly from both sides of the diaper. These flaps or ‘ears’ not only give a fuller coverage to the body (and hence provide a better fit), but also provide a well-defined leg section.

The hourglass configuration as is commonly used today has relatively broad front and rear portions (with wing like protrusions) connected by a crotch section having an absorbent member. When a diaper is worn, it is positioned in such a way that the absorbent section covers the crotch of the wearer and the front section is folded over so as to cover the front portion of the wearer. The rear portion covers the bottom of the wearer. The outward projecting ears of the front portion (from the navel to the side of the body) are joined to the outwardly projecting ears from the rear portion (coming from the back of the body to the front) thereby covering the side hips of the wearer.

In order for the diaper to stay in place on the body of the wearer there should be some way for the rear ears to be fixed to the front portion of the diaper. This is achieved for example by the use of tapes and/or mechanical fasteners. Since tapes and other fasteners have a tendency to gradually loosen their grip during prolonged periods of wearing the diaper, it is desirable to have a configuration of ears and fasteners such that the diaper can be pulled and tightly fitted on the wearer. This would ensure a prolonged period of comfortable wearing.

In order to design better fitting diapers, various approaches have been used. One such approach is the use of elastomeric ears that are attached to the main body of the absorbent article. These ears have elasticity and are joined by means such as welding, adhesives, seams, etc. to the lateral edges of the article. When the article is worn, the ears from the rear side of the article are pulled forward and attached to the front of the body by means of fasteners. The elastomeric nature of the ears helps in achieving the desired fit around the waist of the wearer.

U.S. Pat. No. 5,496,298 to Kuepper et al. discloses the construction of such ears. The ears disclosed are attached to the front and rear portions of the main body of the diaper. The ears are made of elastomeric material (for a better body hugging fit), and are shaped to cover the side of the body (increasing leak protection). Such a configuration results in providing additional available amounts of elastomeric material, especially for the ears, apart from topsheet, backsheet and other components of the article. However, in this design the ears have to be separately cut and then precisely joined to the main body (chassis) of the absorbent article thereby complicating the manufacturing process.

U.S. Pat. No. 4,701,172 to Stevens discloses a stretchable outer cover that is bonded to the body sideliner in its stretched state. This configuration does have flexibility in terms of providing a better fit around the waist of the wearer. However, the elastic members attached to the backsheet require additional inventory and process steps.

U.S. Pat. No. 6,547,773, issued to Kleinschimdt et al., describes an elastic backsheet. However this invention discloses a backsheet that is elastic throughout and not in zones such as the ear portion, etc., thereby increasing the cost of manufacture of the article.

It is therefore desirable to have absorbent articles that provide a good fit to the wearer and require a minimum amount of material for manufacture. The articles should eliminate the need of separate components for manufacture, and also eliminate the requirement for the attachment of various parts during the manufacturing process.

It is accordingly an objective of the present invention to provide for an absorbent article with an integrated chassis with elastomeric characteristics in predetermined regions of the chassis.

It is also an objective of the present invention to provide an article that has fewer components and is easy to manufacture.

### SUMMARY OF THE INVENTION

To achieve the aforementioned objects and to overcome the drawbacks of the prior art, the present invention provides an absorbent article made of composite material. The absorbent article of the invention comprises an absorbent chassis comprising a liquid permeable top sheet, a liquid impermeable back sheet and an absorbent core disposed between the top sheet and the back sheet. The absorbent chassis defines a front waist portion, a back waist portion and a crotch portion that extends between and interconnects the front and back waist portions. The back sheet of the absorbent article has laterally protruding ears in the rear and front waist portions and is made from a single sheet of composite material having elastomeric characteristics in predetermined regions.

In one embodiment, regions such as the ear portion of the absorbent article are allowed to remain elastomeric, whereas the remaining portion of the back sheet is rendered inelastic by treating it with chemicals and/or heat.

### BRIEF DESCRIPTION OF THE DRAWINGS

FIG. 1 shows the top view of an absorbent article with elastomeric ears in accordance with the prior art.

FIG. 2 shows the top view of an absorbent article of the present invention with an integrated chassis having elastomeric ear zones.

FIG. 3 shows an alternate embodiment of the present invention.

FIG. 4 is a front view of the absorbent article in accordance with an embodiment of the present invention.

FIG. 5 is a front view of the absorbent article in accordance with an alternate embodiment of the present invention.

### DETAILED DESCRIPTION OF THE INVENTION

The present invention discloses an hourglass shaped absorbent article having an integrated chassis with selective elastomeric zones. The invention can be better understood with the help of accompanying figures.

In the foregoing specification, specific embodiments of the present invention have been described. However, one of ordinary skill in the art will appreciate that various modifications and changes can be made without departing from the scope of the present invention as set forth in the claims below. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims.

The term “disposable absorbent article” as mentioned herein refers to articles that absorb and contain body discharges. The articles are discarded after a first use and are not intended to be used more than once. The invention is described below with reference to hourglass shaped diapers that are placed close to the body and wrapped over so as to form an underwear-like structure. However it will be understood that the invention is also applicable to other types of absorbent articles such as adult incontinent briefs, children training pants and the like.

The portions of the article can be described using the following terms. If the article is spread out flat on a table with the body-touching portion facing up, then the visible surface is referred to as the ‘top of the article’. The surface touching the table is referred to as the ‘bottom of the article’. The near-rectangular portion in the middle of the article is referred to as the ‘crotch section’. The larger outstretched portion is referred to as the ‘rear of the article’, and more particularly the ‘bottom-rear’ portion of the article covering the buttocks of the wearer. The portion of the article at the other end of the ‘crotch section’ is referred to as the ‘front portion’ of the article. The ‘bottom-front’ portion forms the facing portion of the article.

The invention will now be explained with reference to the accompanying figures. Although reference herein is made to baby diapers, it will be appreciated that the invention is also applicable to all such products that have a waist region, such as training pants, adult incontinence articles and the like. While the present invention is described by reference to diapers that have an hourglass configuration, the invention is also applicable to other kinds of configurations wherein a diaper fit is attained by changing the configuration at the waist, for example pant-type diapers and the like.

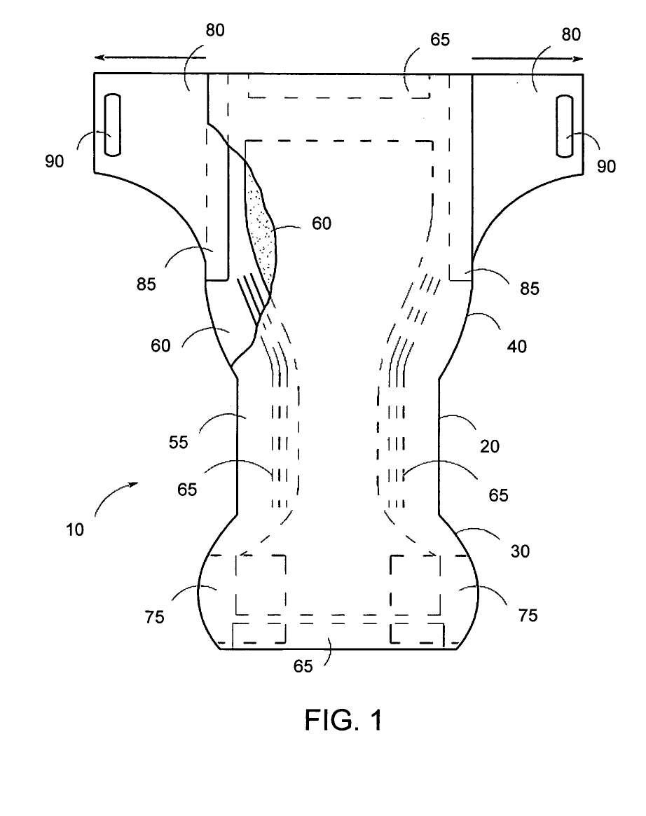

FIG. 1 illustrates an hourglass-shaped absorbent article 10 in accordance with the prior art. Absorbent article 10 has a substantially rectangular crotch portion 20 that interconnects a front portion 30 and a rear portion 40. Front portion 30 and rear portion 40 are positioned adjacent to the shorter edges of crotch portion 20. It may be noted that the front, rear and crotch portions are used herein as merely indicative of various sections of the article. These portions are generally present as one single piece although they have different layers, and various portions of the article may have one or more layers.

A liquid permeable body liner 55 forms a top sheet of absorbent article 10. A liquid impervious sheet 60 forms the bottom sheet of absorbent article 10. Interposed between these sheets is an absorbent core 50 that functions to absorb body fluids. Rear portion 40 spans outwards and has elastomeric members called elastomeric ears 80 at respective left and right edges of absorbent article 10. Elastomeric ears 80 are attached to rear portion 40 at regions 85 using attachment means including adhesives, ultrasonic welding and the like. Elastomeric ears 80 have fasteners 90 attached to their edges. These fasteners are intended to engage coupling member 75 placed on the left and right edges of front portion 30 of absorbent article 10.

Absorbent article 10 is fastened to the wearer such that when article 10 is worn, crotch portion 20 is positioned between the legs of the wearer. Front portion 30 covers the area of the stomach below the navel while rear portion 40 covers the hips of the wearer. Ears 80 are pulled over from behind such that they cover the side hips using fasteners 90 that are joined to coupling member 75 on the front portion of absorbent article 10. Absorbent article 10 is further provided with elastic members 65 at crotch portion 20, front portion 30 and rear portion 40. Fasteners 90 and coupling members 75 ensure that absorbent article 10 may be tightened or loosened depending on the wearer's convenience.

Ear-like projections 80 are formed of elastomeric material and project outwards from the center of absorbent article 10. Since the elastomeric ears are separately cut and then joined to the rest of the article, the manufacturing process becomes complicated. Furthermore the need for maintaining additional inventory also arises.

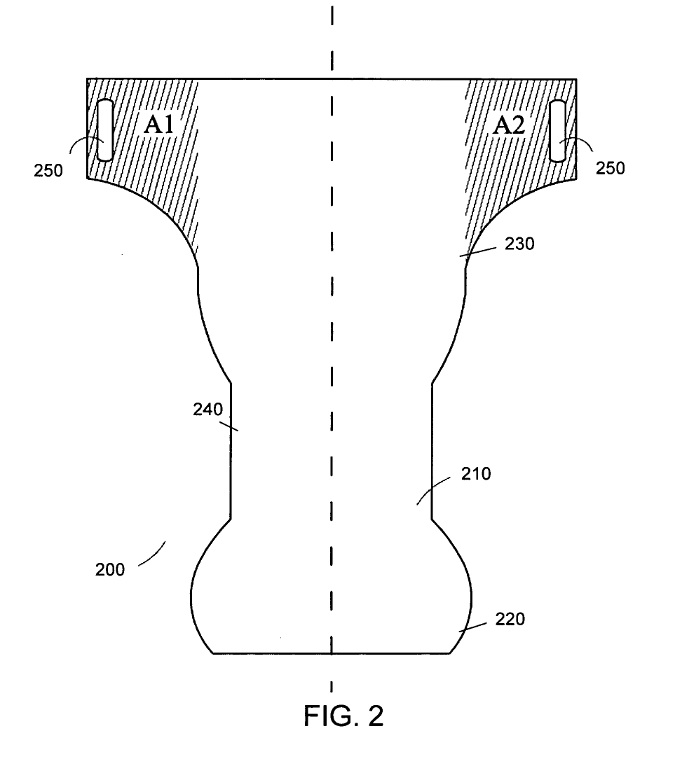

The present invention provides an integrated chassis that has elastomeric properties in certain zones. FIG. 2 shows an absorbent article 200 in accordance with an embodiment of the present invention. Absorbent article 200 has an integrated absorbent chassis 210 that does away with the need for separate components. Integrated absorbent chassis 210 defines a front waist portion 220, a back waist portion 230 and a crotch portion 240 that extends between and interconnects front and back waist portions 220 and 230. Fastener 250 is attached to back waist portion 230 of the absorbent chassis. Fastener 250 may be provided by using conventional means, well known to those skilled in the art, such as adhesive, thermal, cohesive, and ultrasonic bonding.

Integrated chassis 210 comprises a back sheet, an absorbent core and a top sheet that are placed and joined to form an integrated structure. The back sheet is made from a single sheet of composite material that has elastomeric characteristics. The elastomeric characteristics are allowed to remain active in certain zones such as ear portions A1 and A2. The remainder portions of the garment may be treated by heat and/or chemicals to suppress the elastomeric characteristics. The top sheet can be made of non-elastic material. The top sheet is disposed only to cover crotch portion 240 i.e. excluding the areas A1 and A2. This ensures that when the elastomeric ears A1 and A2 are pulled when the article is worn, the elasticity of the ears is not restricted by the non-elastic members.

In an alternative embodiment, the top sheet may be made from a composite material having selective zones with elastomeric characteristics. In this case, both the top sheet and the back sheet may have ear projections that can be kept elastomeric.

However, in both of the embodiments described above, care has to be taken that the material chosen has the desired properties expected of the particular layer. For example, the top sheet has to be soft to the body, permeable and preferably made from a breathable fabric. The back sheet needs to be liquid impermeable, breathable and non slippery.

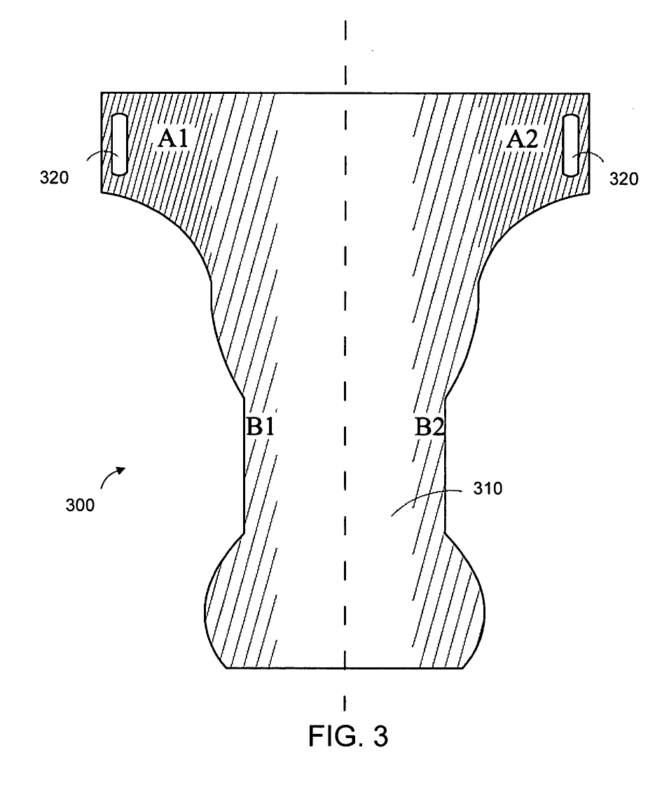

In yet another embodiment of the invention as shown in FIG. 3, absorbent article 300 has an integrated chassis 310, and fastener 320 on chassis 310. In addition to elastomeric ear portions A1 and A2 of integrated chassis 310, the areas B1 and B2 near the longitudinal edges of the crotch portion are also elastic. While wearing the article, this area is positioned on either side of the crotch area of the wearer. The elasticity ensures that the article fits snuggly and leakage does not take place. The leakage prevention may be further enhanced by using means such as side elastics and containment flaps well known in the art.

It will be understood that the elasticity of areas B1 and B2 may differ from that of the elastic ears. This selective elastic stretchability may be achieved by treating various zones with varying amounts of thermal, sonic or chemical treatment.

The materials used for individual components may be chosen from a wide variety of known suitable materials. The terms “elastic” and “stretchable” include any material which can be stretched, and which tends to return to its original shape when relaxed. Suitable polymers for manufacturing individual components include, without limitation, block copolymers of polystyrene, polyisoprene and polybutadiene; copolymers of ethylene, natural rubbers and urethanes; and combinations of the foregoing. Other suitable polymers include copolymers of ethylene, including without limitation ethylene vinyl acetate, ethylene methyl acrylate, ethylene ethyl acrylate, ethylene acrylic acid, stretchable ethylene-propylene copolymers, and combinations thereof. As a general rule, the elastomeric material used in the present invention is stretchable in at least one direction. Preferably, the elastomeric material is stretchable in two directions. When the elastomeric material is stretchable in a single direction, the stretch direction of the elastomeric material will be oriented so as to provide elastomeric forces which tend to pull the front and rear portions of the article towards one another such that the article is maintained at about the waist of a wearer. The elastomeric material can comprise materials such as a stretch-bonded-laminate (SBL) material, a neck-bonded-laminate (NBL) material, an elastomeric film, an elastomeric foam material, or the like. For example, suitable meltblown elastomeric fibrous webs are described in U.S. Pat. No. 4,663,220, issued May 5, 1987, to T. Wisneski et al., the disclosure of which is hereby incorporated by reference in its entirety.

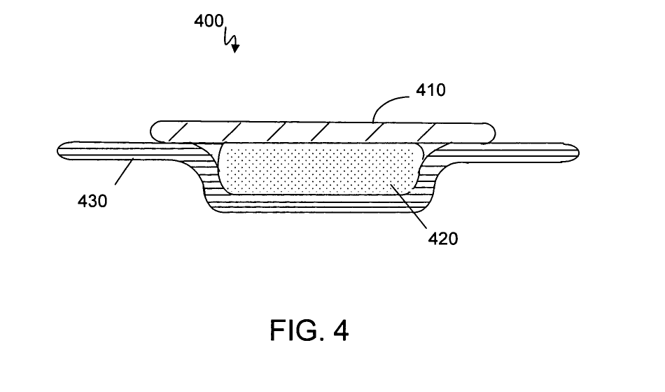

FIG. 4 shows a front view of the absorbent article in accordance with an embodiment of the present invention. Absorbent article 400 has a top sheet 410, an absorbent core 420 and a back sheet 430. Top sheet 410 may be made of non-elastic material, while back sheet 430 is made of a composite material. As shown, top sheet 410 only covers the region directly above absorbent core 420 (crotch portion), and does not extend to cover the laterally extending ear portions (corresponding to areas A1 and A2).

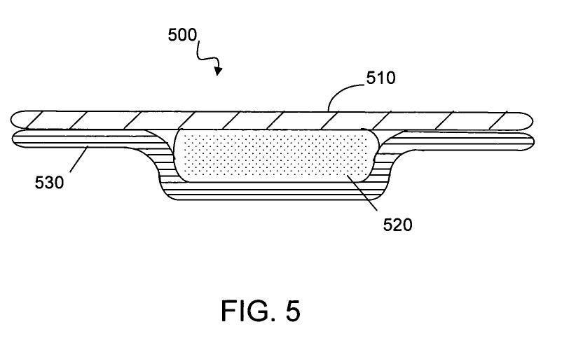

FIG. 5 shows a front view of the absorbent article in accordance with an alternate embodiment of the present invention. Absorbent article 500 has a top sheet 510, an absorbent core 520 and a back sheet 530. Both top sheet 510 and back sheet 520 are made of a composite material and have elastomeric characteristics in the laterally extending ear portions (corresponding to areas A1 and A2).

In various embodiments, the top sheet is formed from material such as fibrous non-woven fabric or porous plastic film. Preferred non-woven materials include spun-bonded polypropylene, spun-bonded polyethylene, and thermally bonded webs of staple fibers. The back sheet is made from material such as plastic film or a laminate sheet of plastic film. The plastic film is preferably air-impermeable. Such material should be hydrophobic, soft in texture, and strong in tensile strength. An example includes hydro-entangled non-woven webs, which may contain some cotton and/or rayon fibers blended in with thermal-plastic fibers. Cellulose fibers can also be blended in at small percentages to reduce cost. Other materials for forming the back sheet may include polypropylene films, co-extruded films (polyethylene and ethylene vinyl acetate), co-polymer films (polyethylene/polypropylene), and polylaminates (polypropylene nonwoven and polyethylene film). The absorbent core can be made up of any suitable absorbent material, as well as combinations of different types of absorbent materials. For example, the absorbent core may be formed from a mixture of pulp fluff and superabsorbent particles wrapped in a liquid permeable tissue wrap. The superabsorbent particles may be substantially homogeneously mixed with the hydrophilic fibers, or may be non-uniformly mixed. The pulp fluff may be exchanged with synthetic, polymeric, meltblown fibers or with a combination of meltblown fibers and natural fibers. Examples of superabsorbent materials include polyacrylamides, polyvinyl alcohol, polyacrylates, various grafted starches, and the like. A desired super absorbent material is a cross-linked polysodium acrylate, which can be purchased from BASF Corporation of Portsmouth, Va., under the trademark ASAP 2260. The absorbent core may be bonded to the top sheet and back sheet with an adhesive or welding. Leg elastics and waist elastics comprise material such as thread- or tape-like rubber, tape-like plastic foam or plastic film.

The aforementioned embodiments and examples are meant to be for explanatory purposes only, and are not meant to be limiting. Various other configurations are possible within the spirit of the invention. For example, the back sheet may be further elasticized in the back rear portion of the article so as to provide better support during wear. The invention intends to cover all those embodiments within the boundaries defined by the appended claims.

What is claimed is:

1. An absorbent article comprising:
an absorbent chassis comprising a liquid permeable top sheet, a liquid impermeable back sheet and an absorbent core disposed between the top sheet and the back sheet, the absorbent chassis defining a front waist portion, a back waist portion and a crotch portion that extends between and interconnects the front and back waist portions;
wherein the back sheet has laterally protruding ears in the rear and front waist portions, and wherein the back sheet is made from a single sheet of material having non-elastomeric and elastomeric characteristics in predetermined regions.

2. The article as claimed in claim 1, wherein the laterally protruding ears in the rear and front waist portions comprise a fastening component.

3. The article as claimed in claim 2, wherein the fastening component is selected from the group consisting of hook and loop fasteners, button fasteners, and tape fasteners.

4. The article as claimed in claim 1, wherein the back sheet has elastomeric characteristics along the ears in the front and rear waist portions.

5. The article as claimed in claim 1, wherein the back sheet has elastomeric characteristics along the edges of the crotch portion.

6. The article as claimed in claim 1, wherein the top sheet is made of composite material and has elastomeric characteristics in one or more regions corresponding to the elastomeric regions of the back sheet.

7. The article as claimed in claim 1, wherein said composite material is selected from the group consisting of stretch-bonded-laminate (SBL) material, neck-bonded-laminate (NBL) material, elastomeric film and elastomeric foam material.

8. The absorbent article as claimed in claim 1 further comprising a pair of leg elastics adapted to fit about the legs of a wearer.

9. The absorbent article as claimed in claim 1 further comprising an elastic material positioned in the waist region of the article adapted to fit about the waist of a wearer.

10. The absorbent article as claimed in claim 1 further comprising containment flaps, the containment flaps helping reduce the leakage of body exudates from the absorbent article.

11. A disposable absorbent article defining a front waist portion, a back waist portion and a crotch portion that extends between and interconnects the front and back waist portions, the disposable absorbent article comprising:
a liquid impermeable back sheet having laterally protruding ears in the rear and front waist portions;
a liquid permeable top sheet;
an absorbent core disposed between the top sheet and the back sheet; and
at least one fastening component attached to the back waist portion of the absorbent chassis;
wherein the back sheet is made from a single sheet of composite material having elastomeric characteristics in predetermined regions.

12. The disposable absorbent article as claimed in claim 1 wherein the top sheet is made from a single sheet of composite material and has elastomeric characteristics in one or more regions corresponding to the elastomeric regions of the back sheet.
